(19) United States
(12) Patent Application Publication
(10) Pub. No.: US 2024/0382684 A1
(43) Pub. Date: Nov. 21, 2024

(54) Syringe Assembly with In-Molded RFID Sleeve

(71) Applicant: Becton Dickinson France, Le Pont-de-Claix (FR)

(72) Inventors: Alfred Leibbrand, Claix (FR); Sébastien Jouffray, Saint Martin D'Uriag (FR)

(21) Appl. No.: 18/693,219

(22) PCT Filed: Sep. 19, 2022
(86) PCT No.: PCT/EP22/75890

§ 371 Date: Mar. 19, 2024

(30) Foreign Application Priority Data
Sep. 20, 2021 (EP) 21306295.3

(51) Int. Cl. A61M 5/31

(52) U.S. Cl. CPC A61M 5/31; A61M 2005/3104; A61M 2205/3592; A61M 2205/60

(57) ABSTRACT

A syringe assembly includes a syringe having a reservoir and a distal tip, a cap component engaged with the distal tip, and an identification sleeve engaged with the cap component via one or more engagement features extending from at least one of the cap component and the identification sleeve. The identification sleeve comprises an RFID tag to allow the traceability of said syringe before final product labelling.

### CROSS-REFERENCE TO RELATED APPLICATION

The present application claims priority to European Patent Application No. 21306295.3, entitled “Syringe Assembly with In-Molded RFID Sleeve”, filed Sep. 20, 2021, the entire disclosure of which is hereby incorporated by reference in its' entirety.

### BACKGROUND OF THE INVENTION

### Field of the Invention

The present disclosure relates to medical injection devices utilizing data tags or inlays, such as radio frequency identification (RFID) tags or inlays

### Description of Related Art

Medical injection devices such as, e.g., pre-fillable or prefilled syringes usually include a hollow body or barrel forming a container for a medical product. This body includes a distal end which optionally may be provided with a needle, and a proximal end, usually provided with a flange.

There is an increasing need for individual traceability of medical containers such as medical injection devices, with such traceability extending from the manufacturing process until the final labeling, final use, and/or disposal of the medical containers.

While it has been known to utilize machine-readable identifiers such as bar codes, QR codes, and/or RFID tags in order to provide traceability of products such as, e.g., medical injection devices, such identifiers are often accessible from the surface of the containers or other devices, making the identifiers potentially subject to damage or tampering. Additionally, the location of many existing identifiers on the devices is often not conducive to effective and efficient scanning/reading.

Furthermore, many existing solutions call for the RFID tags to be attached to various components of the syringes late in the manufacturing process, such as during product labelling. This does not allow for the syringes to be traced during important pre-labelling processes, such as during filling operations.

Still further, existing RFID tags are susceptible to damage and contamination during normal handling of the syringes, and particularly during inspection and cleaning operations. For example, existing RFID tags can be damaged by electric shock at high voltage leak detection stations, or by steam during EtO (ethylene oxide) and other autoclave sterilization procedures.

### SUMMARY

In view of the foregoing, there exists a need for syringe assemblies having improved traceability, as well as additional features to indicate tampering.

Embodiments of the present disclosure are directed to a syringe assembly including a syringe having a reservoir and a distal tip, a cap component engaged with the distal tip, and an identification sleeve engaged with the cap component via one or more engagement features extending from at least one of the cap component and the identification sleeve. The identification sleeve includes an RFID tag.

In some embodiments, the RFID tag is embedded in the identification sleeve.

In some embodiments, the identification sleeve is rotationally locked to the cap component.

In some embodiments, the one or more engagement features includes a rib or spline extending in a longitudinal direction.

In some embodiments, the one or more engagement features are equally spaced about an inside circumference of the identification sleeve.

In some embodiments, the identification sleeve is press fit or glued onto the cap component.

In some embodiments, the identification sleeve includes a flange engaging a proximal face of the cap component to prevent the identification sleeve from sliding relative to the cap component.

In some embodiments, the flange defines an aperture through which the distal tip extends.

In some embodiments, the identification sleeve includes one or more tabs engaging a proximal face of the cap component to prevent the identification sleeve from sliding relative to the cap component. Each of the one or more tabs is configured to deflect radially outward to allow the identification sleeve to slide over the cap component during installation of the identification sleeve onto the cap component.

In some embodiments, each of the one or more tabs includes a ramped surface configured to engage a distal end of the cap component and cause the one or more tabs to deflect radially outward during installation of the identification sleeve onto the cap component.

In some embodiments, the identification sleeve is substantially the same length as the cap component in a longitudinal direction.

In some embodiments, the identification sleeve is longer than the cap component in a longitudinal direction.

In some embodiments, the syringe assembly further includes a cap sealing the distal tip. The identification sleeve at least partially surrounds the cap.

In some embodiments, at least a portion of an outer diameter of the identification sleeve is substantially equal to an outer diameter of the reservoir.

In some embodiments, the syringe assembly further includes a label adhered to the reservoir and to the identification sleeve.

Other embodiments of the present disclosure are directed to an identification sleeve for a syringe assembly. The identification sleeve includes one or more engagement features extending radially inward from an inner surface of the identification sleeve. The one or more engagement features are configured to engage one or more corresponding engagement features on a cap component of the syringe assembly. The identification sleeve includes an RFID tag.

In some embodiments, the RFID tag is embedded in the identification sleeve.

In some embodiments, the one or more engagement features of the identification sleeve rotationally lock the identification sleeve to the cap component.

In some embodiments, the one or more engagement features includes a rib or spline extending in a longitudinal direction.

In some embodiments, the one or more engagement features are equally spaced about an inside circumference of the identification sleeve.

In some embodiments, the identification sleeve is configured to be press fit onto the cap component.

In some embodiments, the identification sleeve includes a flange extending radially inward from a proximal end of the identification sleeve. The flange is configured to engage a proximal face of the cap component to prevent the identification sleeve from sliding relative to the cap component.

In some embodiments, the flange defines an aperture through which a distal tip of the syringe assembly extends.

In some embodiments, the identification sleeve includes one or more tabs configured to engage a proximal face of the cap component to prevent the identification sleeve from sliding relative to the cap component. Each of the one or more tabs is configured to deflect radially outward to allow the identification sleeve to slide over the cap component during installation of the identification sleeve onto the cap component.

In some embodiments, each of the one or more tabs includes a ramped surface configured to engage a distal end of the cap component and cause the one or more tabs to deflect radially outward during installation of the identification sleeve onto the cap component.

Further details and advantages of the various examples described in detail herein will become clear upon reviewing the following detailed description of the various examples in conjunction with the accompanying drawing figures.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a syringe assembly according to one aspect or embodiment of the present disclosure;

FIG. 2 is a perspective view of the syringe assembly of FIG. 1, with an identification sleeve thereof removed for clarity;

FIG. 3 is an exploded perspective view of the syringe assembly of FIG. 1, with the identification sleeve thereof removed for clarity;

FIG. 4 is a perspective view of an identification sleeve according to one aspect or embodiment of the present disclosure;

FIG. 5 is a perspective view of an RFID tag according to one aspect or embodiment of the present disclosure;

FIG. 6 is a perspective view of the identification sleeve of FIG. 4, with the RFID tag of FIG. 5 embedded therein;

FIG. 7 is a side cross-sectional view of a syringe assembly according to one aspect or embodiment of the present disclosure, including the identification sleeve of FIG. 6;

FIG. 8 is a perspective view of an identification sleeve according to one aspect or embodiment of the present disclosure;

FIG. 9 is a side cross-sectional view of a syringe assembly according to one aspect or embodiment of the present disclosure, including the identification sleeve of FIG. 8;

FIG. 10 is a perspective view of an identification sleeve according to one aspect or embodiment of the present disclosure;

FIG. 11 is a side cross-sectional view of a syringe assembly according to one aspect or embodiment of the present disclosure, including the identification sleeve of FIG. 10;

FIG. 12 is a perspective view of a syringe assembly according to one aspect or embodiment of the present disclosure;

FIG. 13 is a side cross-sectional view of the syringe assembly of FIG. 12;

FIG. 14 is a side cross-sectional view of a syringe assembly according to one aspect or embodiment of the present disclosure;

FIG. 15 a perspective view of a syringe assembly, including a label, according to one aspect or embodiment of the present disclosure; and

FIG. 16 is a partially exploded perspective view of the syringe assembly of FIG. 15.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

All numbers used in the specification and claims are to be understood as being modified in all instances by the term “about”. The terms “approximately”, “about”, and “substantially” mean a range of plus or minus ten percent of the stated value. Further, the term “substantially equal” and like terms mean that the compared values or dimensions are within a range of plus or minus ten percent of one another.

The terms “first”, “second”, and the like are not intended to refer to any particular order or chronology, but refer to different conditions, properties, or elements.

As used herein with reference to an injection apparatus such as a syringe, the term “proximal” refers to an end of the apparatus farthest from the outlet, or to a direction toward the end of the apparatus farthest from the outlet. As used herein with reference to an injection apparatus such as a syringe, the term “distal” refers to an end of the device or apparatus closest to the outlet, or to a direction toward the end of the apparatus closest to the outlet.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

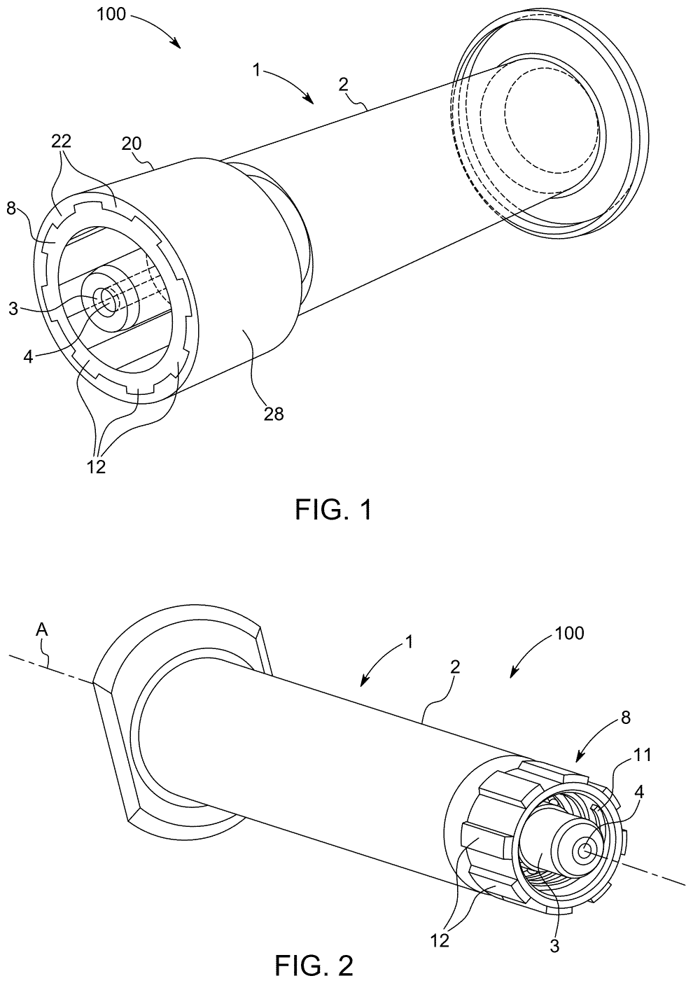

Referring first to FIG. 1, a syringe assembly 100 according to embodiments of the present disclosure includes a syringe 1, a luer lock adapter 8, and an identification sleeve 20. The syringe 1 may include a reservoir 2 integrally connected to a distal tip 3. The reservoir 2 may have a tubular shape and defines a chamber for holding a product, such as a medical fluid. The reservoir 2 and the distal tip 3 may be made a suitable, sterile material, such as a glass or polymer. The reservoir 2 may be sealed at its proximal end by a piston and/or plunger (not shown), that can be actuated distally along a longitudinal axis A of the reservoir 2 to expel the product from the reservoir 2. The distal tip 3 defines a lumen 4 through which the product can be expelled from the reservoir 2. The distal tip 3 may be configured for fluid connection to other components (not shown), such as an intravenous (IV) connector or needle hub, to facilitate sterile transfer of the product from the reservoir 2.

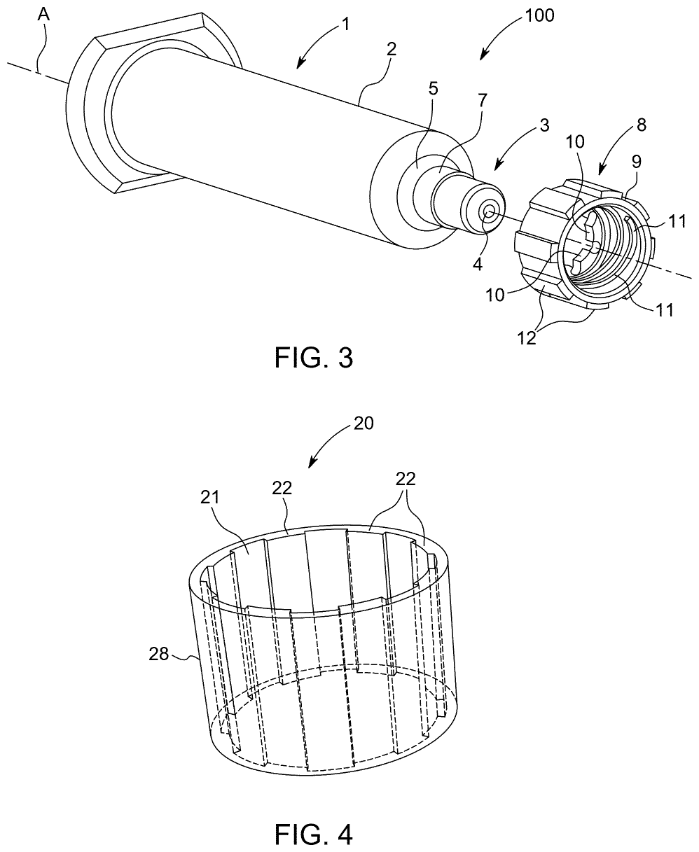

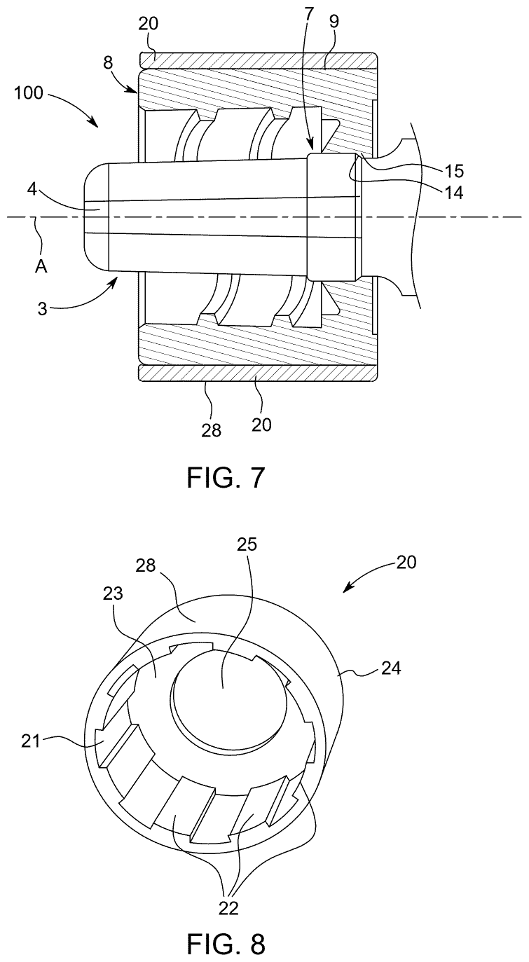

Referring now to FIGS. 2 and 3, the syringe assembly 100 is illustrated with the sleeve 20 removed to more clearly illustrate the features of the luer lock adapter 8. The luer lock adapter 8 includes a generally tubular ring 9 that at least partially surrounds the distal tip 3 of the syringe 1. An annular projection 10 extends radially inward from a proximal end of the ring 9 to engage a coupling portion 7 of the distal tip 3 of the syringe 1. In some embodiments, as shown in FIG. 7, the annular projection 10 may include a lip 15 configured to engage a distal side 14 of the coupling portion 7 of the distal tip 3 to prevent removal of the luer lock adapter 8 from the distal tip 3. In some embodiments, the luer lock adapter 8 may be free to rotate about the longitudinal axis A relative to the distal tip 3. In some embodiments, the luer lock adapter 8 may be rotationally locked to the distal tip 3 by, for example, an interference fit between the annular projection 10 and the coupling portion 7.

With continued reference to FIGS. 2 and 3, an external surface of the ring 9 of the luer lock adapter 8 may include one or more engagement features 12, such a splines or ribs. In some embodiments, the one or more engagement features 12 may include eight splines or ribs. Each of the engagement features 12 may extend generally longitudinally along the ring 9 parallel to the longitudinal axis A. The engagement feature 12 may be equally or asymmetrically spaced about the circumference of the ring 9. An internal surface of the ring 9 may include an internal thread 11 configured to engage a connector (not shown), such as an IV connector or a needle hub, in order to make a sterile connection with the syringe 1 and facilitate transfer of the product from the syringe 1 to the connector or vice-versa. The connector may be provided with an external thread configured to cooperate with the internal thread 11 of the luer lock adapter 8 in order to screw the luer lock adapter 8 onto the connector.

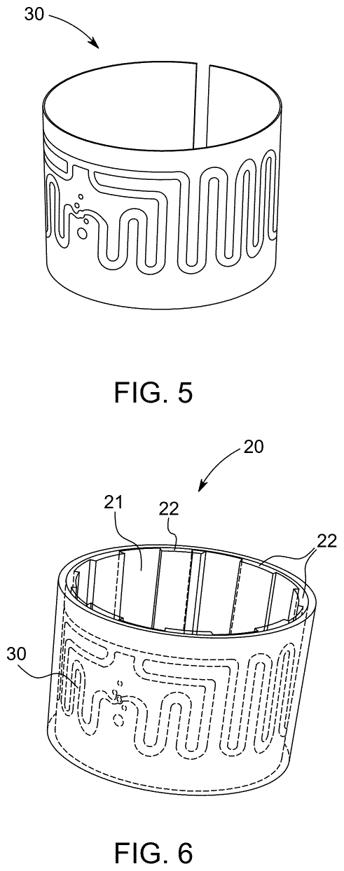

Referring again to FIG. 1 and additionally to FIGS. 4-6, the sleeve 20 may include a radiofrequency identification (RFID) tag 30 (shown in FIGS. 5-6) that can be used to identify and trace the syringe assembly 100 by bringing the syringe assembly into proximity to an RFID scanner (not shown). The sleeve 20 has an internal surface 21 including one or more engagement features 22, such as splines or ribs, extending radially inward toward the longitudinal axis A. Each of the engagement features 22 may extend generally longitudinally along the inner surface 21 of the sleeve 20 parallel to the longitudinal axis A. In some embodiments, the one or more engagement features 12 may include eight splines or ribs. The engagement feature 22 may be equally or asymmetrically spaced about the inside circumference of the sleeve 20. The one or more engagement features 22 of the sleeve 20 may be arranged and configured to engage the one or more engagement features 12 of the adapter 8 to prevent rotation of the sleeve 20 relative to the adapter 8. For example, the one or more engagement features 22 of the sleeve 20 may be arranged to fit between adjacent engagement features 12 of the luer lock adapter 8. In some embodiments, as will be described herein in connection with FIG. 7, the sleeve 20 may have a press fit or interference fit with the luer lock adapter 8 to prevent longitudinal movement of the sleeve 20 relative to the luer lock adapter 8. In some embodiments, the sleeve 20 may be glued to the luer lock adapter 8 to prevent longitudinal movement of the sleeve 20 relative to the luer lock adapter 8. In some embodiments, as will be described herein in connection with FIGS. 8-11, the sleeve 20 may include one or more retaining features to prevent longitudinal movement of the sleeve 20 relative to the luer lock adapter 8. In some embodiments, the sleeve 20 and/or the luer lock adapter 8 may lack their respective engagement features 12, 22, allowing relative rotation between the sleeve 20 and the luer lock adapter 8.

As shown in FIG. 5, the RFID tag 30 may be in the form of a rolled sheet forming a complete or partial circle, though this shaped is only illustrative and other shapes are possible. The RFID tag 30 may be in any form factor such as a laminated inlay or a hard tag encased in resin. In some embodiments as shown in FIG. 6, the RFID tag 30 may be embedded in the sleeve 20 during a molding process. By molding the RFID tag 30 into the sleeve 20, the RFID tag may be protected from damage and contamination during normal handling of the syringe assembly 100, and particularly during inspection and cleaning operations. For example, molding the RFID tag 30 into the sleeve 20 may protect the RFID tag 30 from electric shock at high voltage leak detection stations, and from steam during EtO (ethylene oxide) and other autoclave sterilization procedures. In some embodiments, the sleeve 20 may be formed of a thermoplastic polymer, such as polypropylene or polyethylene, molded at a temperature and pressure sufficiently low so that the RFID tag 30 is not damaged during the molding process. In some embodiments, the sleeve 20 may be extrusion molded over the RFID tag 30. In some embodiments, the RFID tag 30 may be applied to an outer surface 28 or to the inner surface 21 of the sleeve 20 after the sleeve 20 has be molded.

The RFID tag 30 may use any available frequency band, e.g., ultra-high frequency (UHF), high frequency (HF), high frequency-near-field communication (HF-NFC), low frequency (LF), Bluetooth low energy (BLE), and/or a low range wireless (LoRaWan). While RFID tag 30 is shown and described herein, it is to be understood that other forms of remotely writable and readable data tags and/or inlays may be utilized in accordance with the present disclosure. For example, data tags or inlays such as Bluetooth tags, an ultra-wide-band real-time location system (RTLS), a WiFi RTLS, and/or an infrared RTLS may be utilized in lieu of (or in addition to) the RFID tag 30.

Referring now to FIGS. 7-11, various connection mechanisms between the sleeve 20 and the luer lock adapter 8 are illustrated. Referring first to FIG. 7, in some embodiments, the sleeve 20 may be secured to the luer lock adapter 8 via an interference fit (which may also be referred to as a press fit or a friction fit). That is, an inner diameter of the sleeve 20 may be less than an outer diameter of the luer lock adapter 8, such that friction holds the sleeve 20 in place relative to the luer lock adapter 8. In some embodiments, the one or more of the engagement features 22 of the sleeve 20 may have an interference fit between corresponding engagement features 12 of the luer lock adapter 8. In some embodiments, the interference fit between the sleeve 20 and luer lock adapter 8 may be sufficient to prevent the sleeve 20 from sliding off of the luer lock adapter 8 under normal forces associated with handling and use of the syringe assembly 100. In some embodiments, the interference fit between the sleeve 20 and the luer lock adapter 8 may be supplemented by other retaining features, such as described herein in connection with FIGS. 8-11.

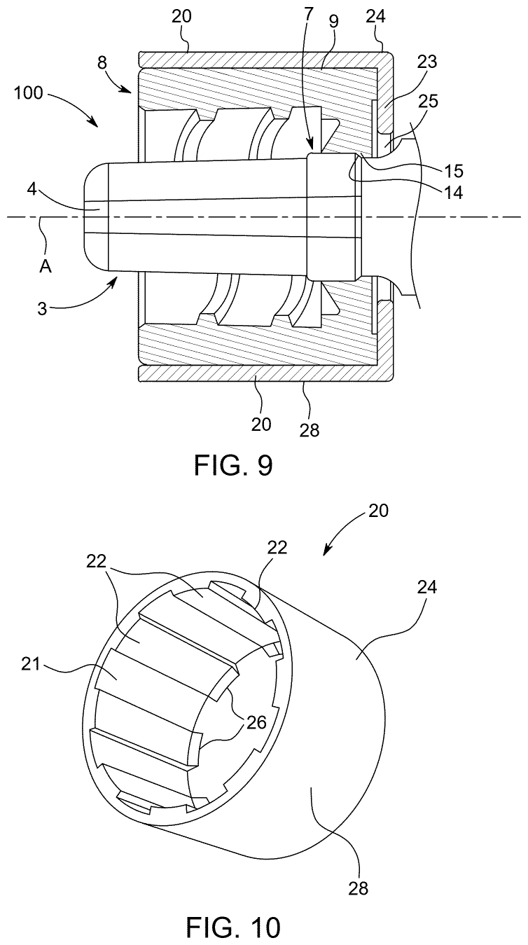

Referring now to FIGS. 8-9, in some embodiments, the sleeve 20 may include a flange 23 extending radially inward from a proximal end 24 of the sleeve 20. The flange 23 may define an aperture 25 large enough that the distal tip 3 of the syringe 1 may extend through the aperture 25, but small enough that the luer lock adapter 8 cannot fit through the aperture 25. As shown, in FIG. 9, the flange 23 abuts a proximal face of the luer lock adapter 8 when installed to prevent the sleeve 20 from sliding distally relative to the luer lock adapter 8. During assembly of the syringe assembly 100, the luer lock adapter 8 may be inserted into the sleeve 20, and the subassembly of the luer lock adapter 8 and the sleeve 20 may then be installed as a unit onto the distal tip 3 of the syringe 1. Alternatively, the sleeve 20 may be positioned in place on the distal tip 3, the luer lock adapter 8 may then be installed onto the distal tip 3 and into the sleeve 20.

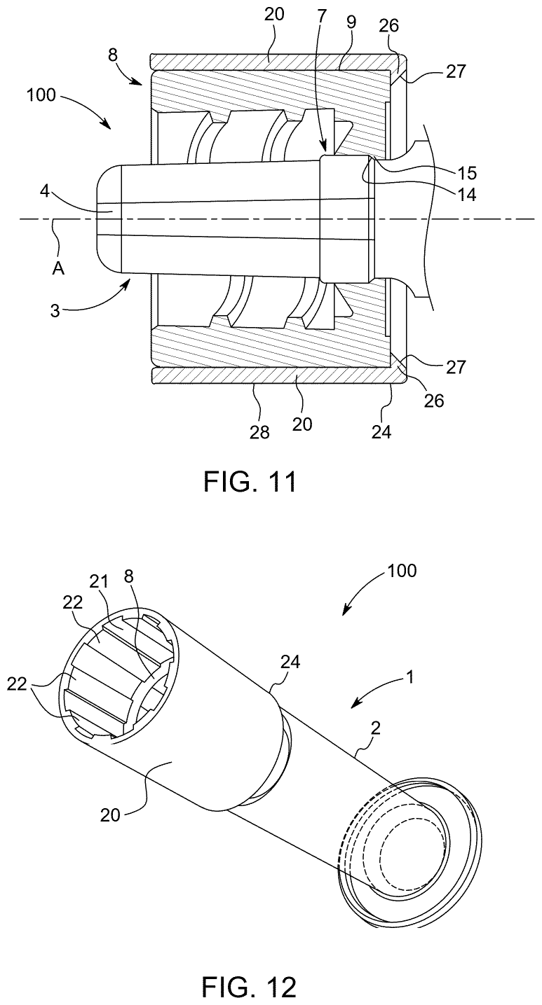

Referring now to FIGS. 10-11, in some embodiments, the proximal end 24 of the sleeve 20 may include one or more tabs 26 configured to engage the proximal face of the luer lock adapter 8. Each of the tabs 26 may include a ramped surface 27 configured to allow installation of the sleeve 20 over the luer lock adapter 8. In particular, the sleeve 20 may be installed with the luer lock adapter 8 already installed on the distal tip 3 by sliding the sleeve 20 in a proximal direction over the luer lock adapter 8. The ramped surface 27 of each tab 26 engages the distal end of the luer lock adapter 8, causing the tabs 26 to deflect radially outward to clear the luer lock adapter 8 and allow the sleeve 20 to slide over the luer lock adapter 8. Once the sleeve 20 is in position such that the tabs 26 are proximal of the luer lock adapter 8, the tabs 26 deflect back inward to engage the proximal face of the luer lock adapter 8. Thus, the tabs 26 facilitate a snap-fit connection between the sleeve 20 and the luer lock adapter 8. With the sleeve 20 installed and the tabs 26 engaging the proximal face of the luer lock adapter 8, the tabs 26 prevent the sleeve 20 from sliding distally relative to the luer lock adapter 8.

In some embodiments, one or more of the various connection mechanisms shown in FIGS. 7-11 may be used in combination with one another to enhance the connection between the sleeve 20 and the luer lock adapter 8.

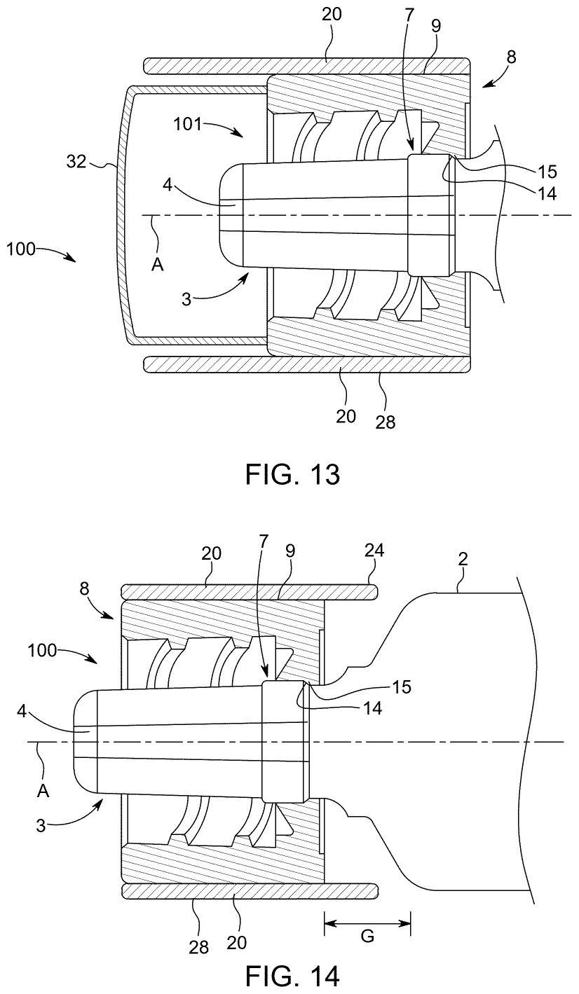

In the embodiments shown in FIGS. 1-11, the sleeve 20 is substantially the same length as the luer lock adapter 8 in a longitudinal direction parallel to the longitudinal axis A. In other embodiments, the sleeve 20 may be longer or shorter than the luer lock adapter 8. For example, as shown in the embodiments of FIGS. 12-13, the sleeve 20 may be longer than the luer lock adapter 8 such that the sleeve 20 extends distally beyond the luer lock adapter 8. The sleeve 20 may at least partially surround a sealing cap 32, e.g. a rigid tip cap (RC) containing an elastomer rigid tip cap (RITC), sealing the distal tip 3 of the syringe 1. Collectively, the luer lock adapter 8 and the sealing cap 32 (including the RC and the RITC) may be referred to as plastic rigid tip cap (PRTC). The sleeve 20 thus prevents removal of the cap 32 unless the sleeve 20 itself is removed or tampered with. Such tampering will be apparent upon visual inspection of the syringe assembly 100, thereby alerting a user to not use the syringe assembly 100.

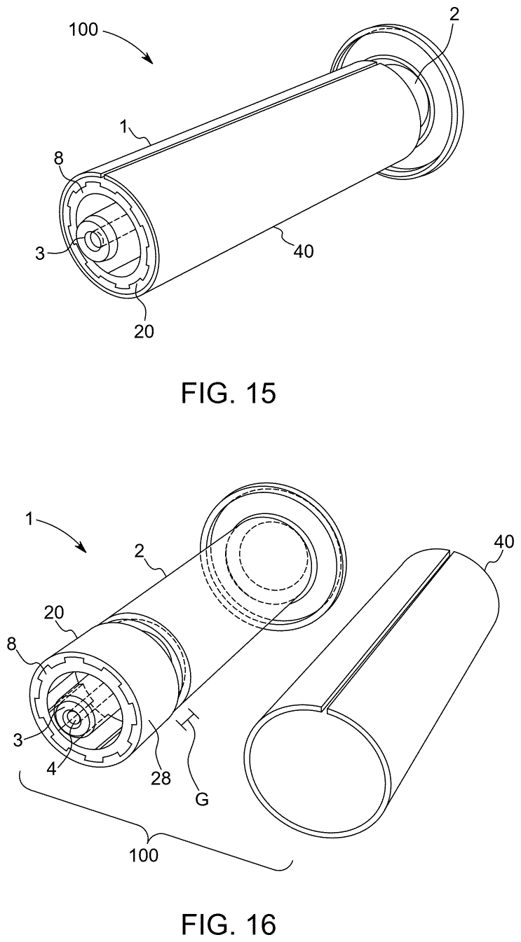

Referring now to FIG. 14, in some embodiments, the sleeve 20 may extend proximally of the luer lock adapter 8. This arrangement reduces a gap G between the sleeve 20 and the reservoir 2, making the syringe assembly 100 easier to handle. Additionally, reduction of the gap G may facilitate more reliable placement of a label over the syringe assembly 100, as will be described herein in connection with FIGS. 15-16. In the embodiments depicted in the accompanying drawings, the proximal end 24 (as shown, for example, in FIGS. 9, 11, and 14) of the sleeve 20 may terminate distally of the reservoir 2, which may be advantageous in that the sleeve 20 does not interfere with visual inspection (either manual or machine) of the syringe 1.

It is noted that the illustrations of FIGS. 12-14 show the sleeve 20 without the flange 23 of FIGS. 8-9 or the tabs 26 of FIGS. 10-11. It is to be understood, however, that one skilled in the art could readily implement the flange 23 and/or tabs 26 in the embodiments shown in FIGS. 12-14, and such variations are to be considered within the scope of the present disclosure.

Referring now to FIGS. 15-16, in some embodiments, a label 40 may be applied to the syringe assembly 100. The label 40 may include an adhesive backing that is attached to the reservoir 2 and the sleeve 20, and extends across the gap G. To facilitate placement of the label 40, at least a portion of an outer diameter of the sleeve 20 may be substantially equal to an outer diameter of the reservoir 2. Additionally, the outer surface 28 of the sleeve 20 may be substantially smooth to promote adhesion and be less susceptible to contamination. The label 40 may extend at least partially around the circumference of the reservoir 2 and the sleeve 20. With the label 40 secured to both the sleeve 20 and the reservoir 2, the label 40 prevents the sleeve 20 from rotating relative to the reservoir 2. Consequently, the luer lock adapter 8 is also prevented from rotating relative to the reservoir 2 due to the engagement between the luer lock adapter 8 and the sleeve 20. This has the advantage that the luer lock adapter 8 is firmly connected to the syringe 1, which increases user confidence that the assembly 100 is secure and sterile. The label 40 may be configured to tear when sufficient rotational force is exerted on the sleeve 20, i.e. when the user twists the sleeve 20 to open the syringe assembly 100, indicating to the user that the syringe assembly 100 has been opened. In some embodiments, the label 40 may also be adhered to the cap 32 (shown in FIG. 14). The label 40 may be configured to tear when the cap 32 is removed, indicating to the user that the syringe assembly 100 has been opened. In some embodiments, the label 40 may include an RFID tag (not shown) to allow for additional monitoring and tracking of the syringe assembly 100.

The sleeve 20 may be installed to the luer lock adapter 8 at various stages in the life of the syringe assembly 100 to enhance traceability of the syringe assembly 100 compared to traditional methods. For example, the sleeve 20 may be connected to the luer lock adapter 8 during an assembly process of the cap 32. Alternatively, the sleeve 20 may be connected to the luer lock adapter 8 after the cap 32 is connected to the syringe 1. These options allow for traceability of the “naked” (i.e. unfilled) syringe assembly 100 using the RFID tag 30. Alternatively, the sleeve 20 may be connected to the luer lock adapter 8 prior to installation of the label 40.

The above-described and illustrated embodiments illustrate the sleeve 20 being used with embodiments of the syringe assembly 100 that include a luer lock adapter 8. However, it is to be understood that the sleeve 20 could also be used with embodiments of the syringe assembly 100 that include a rigid needle shield (RNS). In particular, the one or more engagement features 22 of the sleeve 20 could be configured to engage with one or more engagement features of the RNS, which could be similar to the one or engagement features 12 of the luer lock adapter 8 described herein. As used herein, the term “cap component” means a component of any form or cap for the syringe assembly 100 engaged with the distal tip 3 of the syringe 1, regardless of whether the cap is a PRTC or RNS. In particular embodiments, the term “cap component” may refer to the luer lock adapter 8 or an outer component of an RNS.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A syringe assembly comprising:
a syringe comprising a reservoir and a distal tip;
a cap component engaged with the distal tip; and
an identification sleeve engaged with the cap component via one or more engagement features extending from at least one of the cap component and the identification sleeve;
wherein the identification sleeve comprises an RFID tag.

2. The syringe assembly according to claim 1, wherein the RFID tag is embedded in the identification sleeve.

3. The syringe assembly according to claim 1, wherein the identification sleeve is rotationally locked to the cap component.

4. The syringe assembly according to claim 1, wherein the one or more engagement features comprises a rib or spline extending in a longitudinal direction.

5. The syringe assembly according to claim 1, wherein the one or more engagement features are equally spaced about an inside circumference of the identification sleeve.

6. The syringe assembly according to claim 1, wherein the identification sleeve is press fit or glued onto the cap component.

7. The syringe assembly according to claim 1, wherein the identification sleeve comprises a flange engaging a proximal face of the cap component to prevent the identification sleeve from sliding relative to the cap component.

8. The syringe assembly according to claim 7, wherein the flange defines an aperture through which the distal tip extends.

9. The syringe assembly according to claim 1, wherein the identification sleeve comprises one or more tabs engaging a proximal face of the cap component to prevent the identification sleeve from sliding relative to the cap component,
wherein each of the one or more tabs is configured to deflect radially outward to allow the identification sleeve to slide over the cap component during installation of the identification sleeve onto the cap component.

10. The syringe assembly according to claim 9, wherein each of the one or more tabs comprises a ramped surface configured to engage a distal end of the cap component and cause the one or more tabs to deflect radially outward during installation of the identification sleeve onto the cap component.

11. The syringe assembly according to claim 1, wherein the identification sleeve is substantially the same length as the cap component in a longitudinal direction.

12. The syringe assembly according to claim 1, wherein the identification sleeve is longer than the cap component in a longitudinal direction.

13. The syringe assembly according to claim 1, further comprising a cap sealing the distal tip,
wherein the identification sleeve at least partially surrounds the cap.

14. The syringe assembly according to claim 1, wherein at least a portion of an outer diameter of the identification sleeve is substantially equal to an outer diameter of the reservoir.

15. The syringe assembly according to claim 1, further comprising a label adhered to the reservoir and to the identification sleeve.
